(19) United States
(12) Patent Application Publication
(10) Pub. No.: US 2010/0196432 A1
(43) Pub. Date: Aug. 5, 2010

(54) BIOPOLYMER STRUCTURES

(76) Inventors: Adam W. Feinberg, Cambridge, MA (US); Kevin Kit Parker, Cambridge, MA (US)

Correspondence Address: MCCARTER & ENGLISH, LLP BOSTON, 265 Franklin Street, Boston, MA, 02110, US

(21) Appl. No.: 12/443,890

(22) PCT Filed: Oct. 10, 2007
(86) PCT No.: PCT/US07/21724

§ 371 Date: Apr. 8, 2010

Related U.S. Application Data
(63) Continuation of application No. 60/828,948, filed on Oct. 10, 2006.

(51) Int. Cl. A61K 9/00; A61K 35/12; A23L 1/212; A23L 1/31; C12N 11/02; C07K 14/78; C07K 14/75; C07K 14/435

(52) U.S. Cl. 424/422; 424/93.7; 426/615; 426/641; 435/177; 530/350; 530/353; 530/356; 530/382; 530/395; 536/23.1

(57) ABSTRACT

The invention described herein relates to biopolymer structures. The biopolymer structures are spatially organized from the nanometer to centimeter length scales and incorporate functionally active cells. Applications of the biopolymer structures include use with stem cells.

### CROSS-REFERENCE TO RELATED APPLICATIONS

This application claims priority to U.S. Provisional Patent Application No. 60/828,948, filed Oct. 10, 2006, which is incorporated by reference into this disclosure in its entirety.

### GOVERNMENT SUPPORT

The invention was supported, in whole or in part, by grant Prime Award Number FA9550-01-1-0015 from the Defense Advanced Research Projects Agency under the United States Department of Defense. The Government has certain rights in the invention.

### BACKGROUND

Millions of surgical procedures are performed each year that require tissue or organ substitutes to repair or replace damaged or diseased organs or tissues Such procedures require devices and materials that replicate, augment or extend functions performed by biological systems. Existing scaffolds are limited in their capacity to support growth, differentiation, and function of cells and engineered tissue.

### SUMMARY

The invention provides improved biopolymer structures that overcome the limitation of earlier compositions. The biopolymer structures are spatially organized from the nanometer to centimeter length scales and may incorporate functionally active cells.

Within the invention is a freestanding functional tissue structure containing a flexible polymer scaffold (e.g., biologically derived) that is imprinted with a predetermined pattern and cells attached to said polymer. The cells are spatially organized according to the imprinted pattern, and the cells are functionally active. By functionally active, it is meant that the cell attached to the polymer scaffold comprises at least one function of that cell type in its native environment. For example, a myocyte cell contracts, e.g., a cardiomyocyte cell contracts along a single axis. Neural cells transduce or transmit an electrical signal to another neural cell, muscle cell or other cell type. The tissue structure optionally contains a plurality of scaffolds or films. The construction of the structure is carried out by assembling the scaffolds and then seeding with cells. Alternatively, the structure is assembled in an iterative manner in which a scaffold is made, seeded with cells, and stacked with another scaffold, which in turn is seeded with cells. This seed/stack process is repeated to construct the structure. In some cases different cell types are seeded together or sequentially, e.g., for construction of neural tissue, glial cells are seeded and then neural cells. The predetermined pattern upon which cells attach and the cell type used to seed the film/polymer scaffold depends upon the desired tissue type. For example, smooth muscle cells are used for blood vessels and other internal organs, striated muscle cell (myoblasts) for skeletal muscle tissue, cardiac (cardiomyocytes) for heart tissue. A muscle tissue structure is composed of bundles of specialized cells capable of contraction and relaxation to create movement. In the body, striated or skeletal muscles move bones, smooth muscle lines blood vessels, stomach, digestive tract, and other internal organs, and cardiac muscle make up the myocardium. As an additional example, stem cells are incorporated into the polymer scaffold. Composition and structure of the polymer scaffold contribute to directing the differentiation of the stem cells to one or more differentiated cells types, which then form a functional, engineered tissue such as muscle, skin, blood vessels, etc.

One use of the engineered tissue structures described herein is to repair and/or reinforce the corresponding tissue in a mammal, e.g., an injured or diseased human subject. For example, the cell-seeded films/polymers are used as or in prosthetic devices, tissue implants, and wound dressing. Such wound dressing offer improved healing of lesions that are often difficult to treat, e.g., burns, bedsores, and abrasions. The structures are also useful to repair other tissue defects, e.g., for organ repair due to birth defects such as gastroschisis or defects due to degenerative diseases. Wound dressing compositions are portable and amenable to both hospital (e.g., operating room) use as well as field (e.g., battlefield) use. The films or polymers are packaged wet or dry, e.g., cell scaffold/net alone, net+ cells, or net+ cells+drug (e.g., antibiotic, blood coagulant or anti-coagulant). A net is characterized by a pattern or mesh of filaments or threads. The filaments or threads are organized into a grid structure or are present in an amorphous tangle. The film is peeled away from a support and applied to injured or diseased tissue.

The compositions are also used to manufacture non-natural food products with superior nutritional or flavor compared to the corresponding naturally-occurring product. Such a composition contains a plurality of freestanding tissue structures, each of which comprises a flexible polymer scaffold imprinted with a predetermined pattern. Muscle cells, e.g., bovine skeletal muscle cells, are attached to the polymer in spatially organized manner according to the pattern to yield an edible meat product, the texture and taste of which are distinguished from a naturally-occurring meat. For example, the meat is more tender and flavorful compared to natural beef. Optionally, the structure also contains adipose cells, layers of fatty tissue, or layers of fatty acids between the muscle cells. For example, the cells contain or produce a different fatty acid compared to natural beef or produce an increased amount of a certain fatty acid, e.g., an omega-3 fatty acid, compared to a naturally-occurring meat. In yet another example, the composition has a longer shelf life compared to natural meat or meat from genetically-modified animals.

Similarly, the structures are useful to make bioengineered plant products, e.g., fruits and vegetables, that are not achieved using traditional horticultural methods. Such a composition contains a plurality of freestanding tissue structures, each of which comprises a flexible polymer scaffold imprinted with a predetermined pattern. Plant cells are attached to the polymer in spatially organized manner according to the pattern to yield an edible fruit or vegetable product, the texture and taste of which is distinguished from a naturally-occurring fruit or vegetable. For example, the engineered fruit or vegetable has a better texture, flavor, color, shelf life, or other characteristic compared to naturally-occurring or genetically modified fruits or vegetables grown from seed. For this application, the cells used for seeding the scaffolds are plant cells, i.e., the cells have one or more of the following structures: cell wall, chloroplast, and vacuole.

A method for creating biopolymer structures is carried out by providing a transitional polymer on a substrate; depositing a biopolymer on the transitional polymer; shaping the biopolymer into a structure having a selected pattern on the transitional polymer (poly(N-Isopropylacrylamide); and releasing the biopolymer from the transitional polymer with the biopolymer's structure and integrity intact. The biopolymer is selected from an extracellular matrix protein, growth factor, lipid, fatty acid, steroid, sugar and other biologically active carbohydrates, a biologically derived homopolymer, nucleic acid, hormone, enzyme, pharmaceutical composition, cell surface ligand and receptor, cytoskeletal filament, motor protein, silks, polyprotein (e.g., poly(lysine)) or a combination thereof. For example, the biopolymer is selected from the group consisting of fibronectin, vitronectin, laminin, collagen, fibrinogen, silk or silk fibroin. For example, the biopolymer component of the structure comprises a combination of two or more ECM proteins such as fibronectin, vitronectin, laminin, collagens, fibrinogen and structurally related protein (e.g. fibrin). The deposited structure includes features with dimensions of less than 1 micrometer.

The biopolymer is deposited via soft lithography. For example, the biopolymer is printed on the transitional polymer with a polydimethylsiloxane stamp. Optionally, the process includes printing multiple biopolymer structures with successive, stacked printings. For example, each biopolymer is a protein, different proteins are printed in different (e.g., successive) printings. Alternatively, the biopolymer is deposited via self assembly on the transitional polymer. Exemplary self assembly processes include assembly of collage into fibrils, assembly of actin into filaments, and assembly of DNA into double strands.

In another approach, the biopolymer is deposited via vaporization of the biopolymer and deposition of the biopolymer through a mask onto the transitional polymer. For example, the biopolymer is deposited via patterned photo-cross-linking on the transitional polymer and patterned light photo-cross-links the biopolymer in the selected pattern. The method optionally includes the step of dissolving non-cross-linked biopolymer outside the selected pattern. The patterned light changes the reactivity of the biopolymer via release of a photoliable group or via a secondary photosensitive compound in the selected pattern.

The method includes a step of allowing the biopolymer to bind together via a force selected from hydrophilic, hydrophobic, ionic, covalent, Van der Waals, and hydrogen bonding or via physical entanglement. The biopolymer structure is released by applying a solvent to the transitional polymer to dissolve the transitional polymer or to change the surface energy of the transitional polymer, wherein the biopolymer structure is released into the solvent as a free-standing structure. For example, the biopolymer is released by applying a positive charge bias to the transitional polymer, by allowing the transitional polymer to undergo hydrolysis, or by subjecting the transitional polymer to enzymatic action.

The biopolymer is constructed in a pattern such as a mesh or net structure. Optionally, a plurality of structures are produced, e.g., the method includes a step of stacking a plurality biopolymer structures to produce a multi-layer scaffold.

Following construction of the biopolymer structure, living cells are integrated into or onto the scaffold. For example, living cells are grown in the scaffold to produce three-dimensional, anisotropic myocardium or other replacement organ (e.g., lung, liver, kidney, bladder). In addition to producing functional muscle tissue for human therapeutic purposes, the methods include growing the living cells in the scaffold to produce consumable meat or produce with an engineered composition. In other applications, the living cells are stem cells, further comprising growing the living cells in the scaffold where the structure, composition, ECM type, growth factors and/or other cell types assist in differentiation of stem cells into functional, engineered tissue to produce a replacement tissue or organ.

The method optionally includes a step of wrapping the biopolymer structure around a three-dimensional implant and then inserting the implant into an organism. For example, the biopolymer structure is placed on or in a wound. The latter application is particularly useful in field, e.g., battlefield, use.

The substrate, e.g., metal, ceramic, polymer or a combination thereof, is characterized as having an elastic modulus is greater than 1 MPa. For example, the substrate is selected from a glass cover slip, polystyrene, polymethylmethacrylate, polyethylene terephthalate film, gold and a silicon wafer.

The methods are useful to produce a free-standing biopolymer structure. Such structures are free-standing or free-floating, i.e., they do not require a support or substrate to maintain their shape or structural integrity. Shape and integrity is maintained in the absence of a support substrate. For example, a free-standing biopolymer structure is characterized as having an integral pattern of the biopolymer with repeating features with a dimension of less than 1 mm and without a supporting substrate. Exemplary structures have repeating features with a dimension of 100 nm or less. The free-standing biopolymer structure contains at least one biopolymer selected from the group consisting of extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, and combinations thereof. Alternatively or in addition, the structure comprises at least one conducting polymer selected from poly(pyrrole)s, poly(acetylene)s, poly(thiophene)s, poly(aniline)s, poly(fluorene)s, Poly(3-hexylthiophene), polynaphthalenes, poly(p-phenylene sulfide), and poly(para-phenylene vinylene)s. The free-standing biopolymer structure is contacted with a population of cells and the cells are seeded on the patterned biopolymer. In some cases, the free-standing biopolymer structure comprises an integral pattern of the biopolymer and molecular remnant traces of poly(N-Isopropylacrylamide).

In one configuration, the freestanding functional tissue structure includes a flexible polymer scaffold imprinted with a predetermined pattern and cells attached to the polymer. In this example, the cells are spatially organized according to predetermined pattern, and the cells are functionally active. For example, the cells are muscle cells such as smooth muscle cells, striated muscle cells, or cardiac cells.

Also within the invention is a composition containing a plurality of freestanding tissue structures, each of which contains a flexible polymer scaffold imprinted with a predetermined pattern, muscle cells and adipose cells attached to the polymer. The cells are located in or on the structure in spatially organized manner as determined by the pattern. The structure is in the form of an edible meat product, the texture, taste and/or nutritional content of which meat product is distinguished from a naturally-occurring meat. For example, the engineered meat product is at least 10%, 25%, 50%, 2-f old, 5-fold, 10-fold or more tender compared to a naturally-occurring meat. The engineered meat product contains at least 10%, 25%, 50%, 2-f old, 5-fold, 10-fold or more omega-3 fatty acids compared to a naturally-occurring meat. Preferably, the taste of the product is palatable or even more palatable then naturally-occurring meat.

In another example, the composition comprises a plurality of freestanding tissue structures, each of which contains a flexible polymer scaffold imprinted with a predetermined pattern with plant cells attached to the polymer in spatially organized manner according to the pattern. In this example, the composition is in the form of an edible fruit or vegetable product, the texture, taste and/or nutritional content of the product being distinguished from a naturally-occurring fruit or vegetable.

Free-standing biopolymer structures include an integral pattern of the biopolymer with repeating features having a dimension of less than 1 mm (e.g., a dimension of 100 nm or less) and functions as a supporting frame during tissue formation. The structure contains an integral pattern of the biopolymer having repeating features with a dimension of less than 1 mm, e.g., less than 100 nm, and embedded within a 3-dimensional gel. As described above, the structure contains at least one biopolymer selected from extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, and combinations thereof. Cells are seeded on the patterned biopolymer before being embedded within a gel. Optionally, the structure contains cells mixed in with a gel precursor and thus become trapped within the gel when the gel is polymerized around the patterned biopolymer. Alternatively, the cells are seeded after the patterned biopolymer is embedded within a gel. The biopolymer structure is embedded in a gel that comprises at least one biological hydrogel selected from fibrin, collagen, gelatin, elastin and other protein and/or carbohydrate derived gels or synthetic hydrogel selected from polyethylene glycol, polyvinyl alcohol, polyacrylamide, poly(N-isopropylacrylamide), poly(hydroxyethyl methacrylate) and other synthetic hydrogels, and combinations thereof.

The compositions described herein are distinguished from other engineered tissues by virtue of the compounds in the underlying scaffold structure (identity of the polymer) and the pattern or architecture of the structure (grid, net, web, etc.). Both aspects are detected by staining using detectable labeling reagents such as antibodies or other ligands that specifically bind to the compositions used to construct the structure. Detection is accomplished using standard techniques such as electron, fluorescent and/or atomic force microscopy.

### BRIEF DESCRIPTION OF THE DRAWINGS

FIG. 1 illustrates a transitional polymer [for example, Poly(N-Isopropylacrylamide (PIPAAm))] coated onto a glass cover slip serving as a rigid substrate.

FIG. 2 illustrates a biopolymer (e.g., the extracellular matrix protein, fibronectin) microcontact printed onto PIPAAm using polydimethylsiloxane (PDMS) stamps in a pattern dictated by the microstructures on the stamp.

FIG. 3 shows an example of a printed biopolymer pattern on a transitional polymer.

FIG. 4 illustrates a free fibronectin scaffold or structure released as an integral structure with its shape preserved and suspended in solution.

FIGS. 5-10 illustrate representative examples of the types of biopolymer scaffolds that may be generated using the described methods herein.

FIG. 11 shows an image of patterned lines of fibronectin on PIPAAm after a two-step stamping process.

FIGS. 12-19 show sequential, time-lapse images for the release of a fibronectin extracellular (ECM) net based biopolymer scaffold from PIPAAm.

FIGS. 20-23 show examples of fibronectin structures after release from PIPAAm.

FIG. 24 shows an example of a fibronectin ECM Net after release from PIPAAm, imaged at 20× magnification under phase contrast, bright field illumination.

FIGS. 25-33 illustrate a sequential, time-lapse series showing the release of patterned lines of fibronectin and cardiomyocytes from PIPAAm.

FIG. 34 demonstrates that the myocyte patterned into lines using a biopolymer scaffold, is capable of generating functional myocyte constructs. Two frames are illustrated from a video of a few aligned, connected and contracting myocyte in (A) systole and (B) diastole.

FIGS. 36-41 show an example of a biopolymer scaffold created by micropatterning laminin (horizontal lines) and fibronectin (vertical lines) on PIPAAm spin coated on glass cover slips.

FIGS. 42 and 42 show an example wherein a biopolymer scaffold design is used to create a monolayer thick anisotropic two-dimensional myocardium that directs the self assembly of cardiomyocytes along a single axis.

FIG. 44 shows an example of a single strand of a biopolymer scaffold suspended across a hole cut into a PDMS film and with cardiomyocytes cultured thereon.

FIG. 45 shows a magnified view of the single strand of FIG. 44.

FIG. 46 shows examples of biopolymer scaffolding embedded in gels.

### DETAILED DESCRIPTION

Free-standing biopolymer structures that are spatially organized from the nanometer to centimeter length scales can be generated via methods described herein. In this context, “biopolymer” refers to any proteins, carbohydrates, lipids, nucleic acids or combinations thereof, such as glycoproteins, glycolipids, proteolipids, etc. These biopolymers are deposited onto a transitional polymer surface using patterning techniques that allow for nanometer-to-millimeter-to-centimeter-scale spatial positioning of the deposited biopolymers. These patterning techniques include but are not limited to soft-lithography, self-assembly, vapor deposition and photolithography, each of which is further discussed, below.

Once on the surface, inter-biopolymer interactions attract the biopolymers together such that they become bound together. These interactions may be hydrophilic, hydrophobic, ionic, covalent, Van der Waals, hydrogen bonding or physical entanglement depending on the specific biopolymers involved. In the appropriate solvent, dissolution or a change in the surface energy of the transitional polymer releases the patterned biopolymer structure from the surface into solution as an integral, free-standing structure. This biopolymer structure can then be used for a variety of applications, a subset of which is listed, below.

In the context of conducted proof-of-concept experiments, structures of the extracellular matrix protein (ECM), fibronectin, were fabricated into free-standing net-like (mesh) structures. Termed, “ECM Nets,” for their appearance, the fibronectin was patterned using microcontact printing onto a less-than-1-μm-thick layer of poly(N-Isopropylacrylamide) (PIPAAm) supported by a glass cover slip. The fibronectin patterned, PIPAAm coated cover slip was placed in an aqueous medium at room temperature; the aqueous medium hydrated and dissolved the PIPAAm layering, causing the release of the ECM Net into solution. Traces of the PIPAAm may remain on the ECM Net and can be detected, e.g., via mass spectrometry, to provide an indication of an ECM Net produced via this method. The micro-pattern of the ECM Net can also be detected as a mode of determining source.

The exact spatial structure of the ECM net can be changed by altering the features of the polydimethylsiloxane (PDMS) stamp used for microcontact printing and/or by printing multiple times at different angles. While substantially orthogonal net structures are principally described and illustrated herein, other patterns (e.g., fractal, radially extending and/or branching) can also be produced. As an example, the pattern can include shapes that match those of a neuron, as illustrated in FIG. 35.

The potential applications of the technology are widespread. For example, the ability to create ECM nets enable the building of three-dimensional tissue engineering scaffolds with nanometer scale (e.g., between 5 nanometers and 1 micron) spatial control by stacking two-dimensional biopolymer sheets into a three-dimensional structure. As used herein, “two-dimensional” structures include a single layer of the basic structure (e.g., scaffold), which can have a thickness of about 5 to 500 nm (e.g., 10, 25, 50, 100, 200, 300, 400, 400 or more nm); whereas “three-dimensional” structures include multiple, stacked layers of the basic structure. Integration of living cells into these biopolymer scaffolds before release, during stacking or afterward will then allow the generation of tissues with a level of spatial control that exceeds current gel, random mesh and sponge structures used. A detailed listing of materials, methods and many potential applications are listed below.

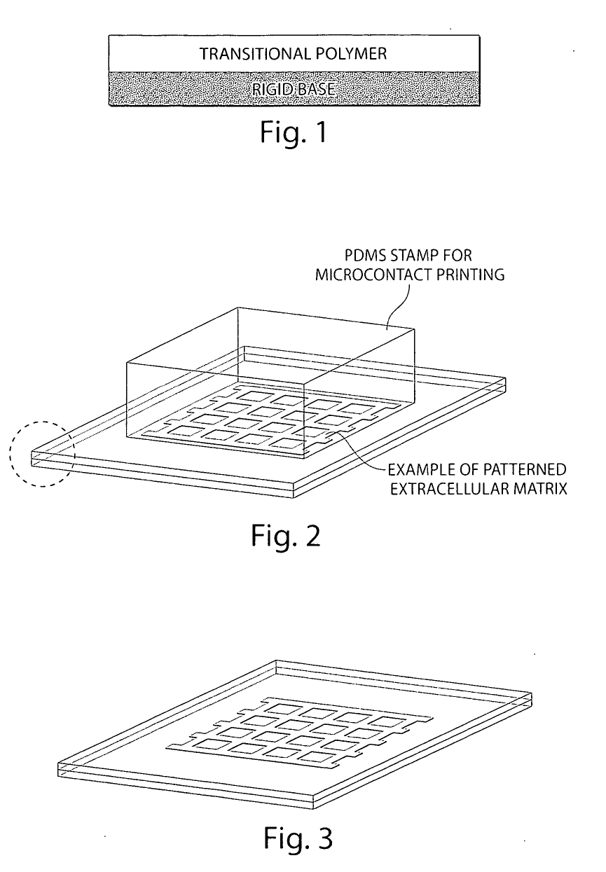

As shown in FIG. 1, a transitional, sacrificial polymer layer is coated on a rigid substrate to form a laminate structure; and a biopolymer scaffold is printed on the sacrificial polymer layer, in this case, using a PDMS stamp for microcontact printing, as shown in FIG. 2.

Materials

The rigid substrate can be any rigid or semi-rigid material, selected from, e.g., metals, ceramics, polymers or a combination thereof. In particular embodiments, the elastic modulus of the substrate is greater than 1 MPa. Further, the substrate can be transparent, so as to facilitate observation during biopolymer scaffold release. Examples of suitable substrates include a glass cover slip, polymethylmethacrylate, polyethylene terephthalate film, silicon wafer, gold, etc.

The transitional, sacrificial polymer layer can be coated onto the substrate. In one embodiment, the transitional polymer is a thermally sensitive polymer that can be dissolved to cause the release of a biopolymer scaffold printed thereon. An example of such a polymer is linear, non-cross-linked poly(N-Isopropylacrylamide), which is a solid when dehydrated, and which is a solid at 37° C. (wherein the polymer is hydrated but relatively hydrophobic). However, when the temperature is dropped to less to 32° C. or less (where the polymer is hydrated but relatively hydrophilic), the polymer becomes a liquid, thereby releasing the biopolymer scaffold.

In another embodiment, the transitional polymer is a thermally sensitive polymer that becomes hydrophilic, thereby releasing a hydrophobic scaffold coated thereon. An example of such a polymer is cross-linked poly(N-Isopropylacrylamide), which is hydrophobic at 37° C. and which is hydrophilic at 32° C.

In yet another embodiment, the transitional polymer is an electrically actuated polymer that becomes hydrophilic upon application of an electric potential to thereby release a hydrophobic (or less hydrophilic) structure coated thereon. Examples of such a polymer include poly(pyrrole)s, which are hydrophobic when oxidized and hydrophilic when reduced. Other examples of polymers that can be electrically actuated include poly(acetylene)s, poly(thiophene)s, poly(aniline)s, poly(fluorene)s, poly(3-hexylthiophene), polynaphthalenes, poly(p-phenylene sulfide), and poly(para-phenylene vinylene)s, etc.

In still another embodiment, the transitional polymer is a degradable biopolymer that can be dissolved to release a structure coated thereon. In one example, the polymer (e.g., polylactic acid, polyglycolic acid, poly(lactic-glycolic) acid copolymers, nylons, etc.) undergoes time-dependent degradation by hydrolysis. In another example, the polymer undergoes time-dependent degradation by enzymatic action (e.g., fibrin degradation by plasmin, collagen degradation by collagenase, fibronectin degradation by matrix metalloproteinases, etc.).

Finally, a spatially engineered surface chemistry is produced on the transitional polymer layer. The surface chemistry can be selected from the following group:
(a) extracellular matrix proteins to direct cell adhesion and function (e.g., collagen, fibronectin, laminin, etc.);
(b) growth factors to direct cell function specific to cell type (e.g., nerve growth factor, bone morphogenic proteins, vascular endothelial growth factor, etc.);
(c) lipids, fatty acids and steroids (e.g., glycerides, non-glycerides, saturated and unsaturated fatty acids, cholesterol, corticosteroids, sex steroids, etc.);
(d) sugars and other biologically active carbohydrates (e.g., monosaccharides, oligosaccharides, sucrose, glucose, glycogen, etc.);
(e) combinations of carbohydrates, lipids and/or proteins, such as proteoglycans (protein cores with attached side chains of chondroitin sulfate, dermatan sulfate, heparin, heparan sulfate, and/or keratan sulfate); glycoproteins [e.g., selectins, immunoglobulins, hormones such as human chorionic gonadotropin, Alpha-fetoprotein and Erythropoietin (EPO), etc.]; proteolipids (e.g., N-myristoylated, palmitoylated and prenylated proteins); and glycolipids (e.g., glycoglycerolipids, glycosphingolipids, glycophosphatidylinositols, etc.);
(f) biologically derived homopolymers, such as polylactic and polyglycolic acids and poly-L-lysine;
(g) nucleic acids (e.g., DNA, RNA, etc.);
(h) hormones (e.g., anabolic steroids, sex hormones, insulin, angiotensin, etc.);
(i) enzymes (types: oxidoreductases, transferases, hydrolases, lyases, isomerases, ligases; examples: trypsin, collegenases, matrix metallproteinases, etc.);
(j) pharmaceuticals (e.g., beta blockers, vasodilators, vasoconstrictors, pain relievers, gene therapy, viral vectors, anti-inflammatories, etc.);
(k) cell surface ligands and receptors (e.g., integrins, selectins, cadherins, etc.); and
(l) cytoskeletal filaments and/or motor proteins (e.g., intermediate filaments, microtubules, actin filaments, dynein, kinesin, myosin, etc.).

Methods

1) Patterning

The rigid substrate can be coated with a thin layer of the transitional polymer by a variety of methods, including spin coating, dip casting, spraying, etc. A biopolymer is then patterned onto the transitional polymer with spatial control spanning the nanometer-to-micrometer-to-millimeter-to-centimeter-length scales. This level of spatial control can be achieved via patterning techniques including but not limited to soft lithography, self assembly, vapor deposition and photolithography. Each of these techniques is discussed, in turn, below.

a) Soft Lithography

In soft lithography, structures (particularly those with features measured on the scale of 1 nm to 1 μm) are fabricated or replicated using elastomeric stamps, molds, and conformable photomasks. One such soft lithography method is microcontact printing using a polydimethylsiloxane stamp. Microcontact printing has been realized with fibronectin, laminin, vitronectin and fibrinogen and can be extended to other extracellular matrix proteins including, but not limited to collagens, fibrin, etc. Other biopolymers can be used as well, as this soft lithography method is quite versatile. There are few, if any, limitations on the geometry of the biopolymer structure(s) beyond the types of patterns that can be created in the polydimethylsiloxane stamps used for microcontact printing. The range of patterns in the stamps, in turn, is presently limited only by the current microprocessing technology used in the manufacture of integrated circuits. As such, available designs encompass nearly anything that can be drafted in modern computer-aided-design software. Multiple layers of biopolymers can be printed on top of one another using the same or different stamps with the same or different proteins to form an integrated poly-protein (poly-biopolymer) layer that can subsequently be released and used.

b) Self Assembly

Various biopolymers will spontaneously form self-assembled structures. Examples, without limitation, of self assembly include assembly of collagen into fibrils, assembly of actin into filaments and assembly of DNA into double strands and other structures depending on base-pair sequence. The self assembly can be directed to occur on the transitional layer to create a nanometer-to-millimeter-centimeter-scale spatially organized biopolymer layer. Further, self assembly can be combined with soft lithography to create a self-assembled layer on top of a soft lithographically patterned biopolymer; alternatively, the processes can be carried out in the reverse order. The self-assembled biopolymer, depending on the strength and stability of intermolecular forces, may or may not be stabilized using a cross-linking agent (for example, glutaraldehyde, formaldehyde, paraformaldehyde, etc.) to maintain integrity of the biopolymer layer upon release from the transitional layer. Otherwise, existing intermolecular forces from covalent bonds, ionic bonds, Van der Waals interactions, hydrogen binding, hydrophobic/hydrophilic interactions, etc., may be strong enough to hold the biopolymer scaffold together.

c) Vapor Deposition

Using a solid mask to selectively control access to the surface of the transitional polymer, biopolymers can be deposited in the accessible regions via condensation from a vapor phase. To drive biopolymers into a vapor phase, the deposition is performed in a controlled environmental chamber where the pressure can be decreased and the temperature increased such that the vapor pressure of the biopolymer approaches the pressure in the environmental chamber. Biopolymer surfaces produced via vapor deposition can be combined with biopolymer surfaces created by self-assembly and/or by soft lithography.

d) Patterned Photo-Cross-linking

Patterned light, x-rays, electrons or other electromagnetic radiation can be passed through a mask by photolithography; alternatively, the radiation can be applied in the form of a focused beam, as in stereolithography or e-beam lithography, to control where the transitional polymer biopolymers attach. Photolithography can be used with biopolymers that intrinsically photo-cross-link or that change reactivity via the release of a photoliable group or via a secondary photosensitive compound to promote cross-linking or breaking of the polymer chains so that the surface areas that are exposed to light are rendered either soluble or insoluble to a developing solution that is then applied to the exposed biopolymer to either leave only the desired pattern or remove only the desired pattern. The biopolymer is provided in an aqueous solution of biopolymer intrinsically photosensitive or containing an additional photosensitive compound(s).

Examples of photo-cross-linking process that can be utilized include (a) ultra-violet photo-cross-linking of proteins to RNA [as described in A. Paleologue, et al., “Photo-Induced Protein Cross-Linking to 5S RNA and 28-5.8S RNA within Rat-Liver 60S Ribosomal Subunits,” Eur. J. Biochem. 149, 525-529 (1985)]; (b) protein photo-cross-linking in mammalian cells by site-specific incorporation of a photoreactive amino acid [as described in N. Hino, et al., “Protein Photo-Cross-Linking in Mammalian Cells by Site-Specific Incorporation of a Photoreactive Amino Acid,” Nature Methods 2, 201-206 (2005)]; (c) use of ruthenium bipyridyls or palladium porphyrins as photo-activatable crosslinking agents for proteins [as described in U.S. Pat. No. 6,613,582 (Kodadek et al.)]; and (d) photocrosslinking of heparin to bound proteins via the cross-linking reagent, 2-(4-azidophenylamino)-4-(1-ammonio-4-azabicyclo[2,2,2]oct-1-yl)-6-morpho-lino-1,3,5-triazine chloride [as described in Y. Suda, et al., “Novel Photo Affinity Cross-Linking Resin for the Isolation of Heparin Binding Proteins,” Journal of Bioactive and Compatible Polymers 15, 468-477 (2000)].

2) Biopolymer Release and Scaffold Formation

The transitional polymer layer dissolves or switches states to release the biopolymer structure(s). For example, a transitional polymer layer formed of PIPAAm (non-cross-linked) will dissolve in an aqueous media at a temperature less than 32° C. In another example, a transitional polymer layer is formed of PIPAAm (cross-linked) will switch from a hydrophobic to hydrophilic state in an aqueous media at a temperature less than 32° C. The hydrophilic state will release the biopolymers. In yet another embodiment, the transitional polymer layer includes a conducting polymer, such as polypyrrole, that can be switched from a hydrophobic to hydrophilic state by applying a positive bias that switches the conducting polymer from a reduced to oxidized state. In additional embodiments, the transitional polymer layer can include a degradable polymer and/or biopolymer that undergoes time-dependent degradation by hydrolysis (as is the case, for example, for polylactic and polyglycolic acid) or by enzymatic action (for example, fibrin degradation by plasmin).

These biopolymer structure(s) can then be further manipulated for the desired application. For example, two-dimensional biopolymer scaffolds can be stacked to form a three-dimensional structure. In another example, the two-dimensional biopolymer scaffolds are seeded with cells before or after release from the transitional polymer before or after stacking to produce a three-dimensional structure. The applications, described below, provide additional details and examples.

Applications

Two-dimensional biopolymer sheets fabricated with nanometer spatial control can be stacked to build a three-dimensional tissue-engineering scaffold. Integration of living cells into these biopolymer scaffolds then allows the generation of tissues with a level of spatial control that extends from the micrometer scale to the meter scale (e.g., between 1 μm and 1 m) and that exceeds the spatial control provided in current gel, random mesh and sponge structures in use, or in other structured scaffolds. Examples of utility include a wide array of tissue-engineering applications. Examples of products and procedures that can be produced with the scaffolds include the following: (a) three-dimensional, anisotropic myocardium used to repair infarcts, birth defects, trauma and for bench top drug testing; (b) spinal cord repair using neuron-specific ECM patterning and growth factors to enhance axonal growth within the central and/or peripheral nervous systems; (c) engineered capillary beds for accelerating and augmenting angiogenesis in tissue-engineered constructs, autologous tissue grafts and traumatically injured tissue; and (d) any of the major organs that require microscale structure for function including but not limited to kidneys, liver, lungs, intestines, visual system, auditory system, nervous systems, muscle, etc.

In another application, two-dimensional scaffolds are wrapped around a three-dimensional object to create patterned surfaces that have nanometer-to-millimeter-to-centimeter-scale features and that cannot be patterned directly using any other technique. This technique is suitable for patterning the surfaces of medical implants to enhance integration with patient anatomy and physiology, such as breast implants, orthopedic implants, dental implants, etc. This technique also is suitable for patterning the surface of artificial vascular grafts to improve re-endothelialization and to hinder smooth muscle growth and intimal hyperplasia.

In another application, ECM fragments (i.e., particles) with defined nanometer, micrometer, millimeter and/or centimeter structure can be inserted as a filler material into wounds to enhance healing by providing an ECM that does not have to be synthesized by fibroblasts and other cells, thereby decreasing healing time and reducing the metabolic energy requirement to synthesize new tissue at the site of the wound.

In another embodiment, the scaffolds can be used as microstructured wound dressings (after cutting the scaffold into a size and shape to fit the wound) that can control the growth direction and morphology of specific cell types based on organization of ECM proteins in a linear and parallel orientation, for example, to maintain myocyte uni-axial alignment in the re-growth of muscle (smooth, cardiac and skeletal), to orient keratinocytes and other epidermal cells to minimize scar formation, and/or to guide axonal growth in peripheral and central nerve regeneration. The scaffold can also be seeded with functional elements, such as drugs, coagulants, anti-coagulants, etc., and can be kept, e.g., in a medic's field pack.

In yet another embodiment, the scaffold can be used to produce engineered food items with unique characteristics that are not found naturally. This technique utilizes the three-dimensional tissue engineering scaffold technology described, above, to generate animal and/or plant tissue where the microstructure and microbiology has been modified in order to change the properties to improve the functionality, nutrition, taste and/or other properties of food stuffs.

For example, this technique can be used to produce designer meat, grown from any type of standard skeletal muscle cells from common animals, such as bovine, swine, or avian, but with certain modifications that make the engineered meat worth the obvious increase in cost associated with a tissue-engineered product. Examples of such modifications include the following: (a) precise control of fat deposits (marbling in beef) to enhance taste, tenderness, etc.; (b) modifications of biopolymer scaffolds and/or genetic modification of myoblasts, such that typical fatty acids in, for example, bovine meat are replaced with healthy fats like the omega-3 fatty acids found in salmon; and (c) merging myoblasts from different animals, such as bovine and swine, creating entirely new meats with taste and texture not previously known. In other examples, designer fruits and vegetables can be modified, similarly to the way the meats, above, are modified, by controlling the amounts of sugars, by adding specific vitamins or minerals and/or by blending different fruits and vegetables to create new hybrids not possible with current horticulture techniques

In another embodiment, the scaffold can be seeded with spray-dried cellular forms, as described in PCT/US2006/031580; this application is incorporated herein by reference in its entirety.

In another embodiment, the scaffold can be seeded with stem cells where the scaffold composition and structure directs (with or without other environmental factors) directs the differentiation. This includes any type of stem cell including embryonic, fetal, neonatal and adult ages. Also includes stem cells with various differentiation capacity including progenitor, multi-potent and pluripotent cells. Stem cell origin may be of an existing cell line, harvested directly from embryos or fetuses, harvested directly from adults, or retrieved from tissue sample/biopsy. In the scaffold, structure, composition, ECM type, growth factors and/or other cell types assist in directing differentiation of stem cells into differentiated cells, thus producing a functional, engineered tissue. The type of engineered tissue created using the stem cells comprises any tissue/organ system in the body (e.g., muscle, nerve, bone, heart, blood vessels, skin, etc.).

In another embodiment, the biopolymer scaffold can be embedded within a gel material to provide spatially patterned chemical, topographical and/or mechanical cues to cells. The biopolymer scaffold is constructed, as has been described, as either a single layer, or as a stacked, 3-D layered structure. A liquid, gel-precursor is then poured around the biopolymer scaffold, and then polymerized (i.e., crosslinked) into a gel. In such a case, cells can either be seeded onto the biopolymer scaffold before embedding in the gel, mixed in with the gel-precursor solution before pouring around the biopolymer scaffold and crosslinking, or seeded onto the combined construct of the biopolymer scaffold embedded in the gel. Examples of gels that can be used include but are not limited to biological gels such as fibrin, collagen, gelatin, etc. and synthetic polymer hydrogels such as polyethylene glycol, polyacrylamide, etc. For example, a nerve graft can be tissue engineered by generating a biopolymer scaffold consisting of a parallel array of long fibronectin strands (such as 20 micrometers wide, 1 centimeter long), seeding neurons on the fibronectin strands, culturing the neurons so they can adhere and grow along the fibronectin, embed the fibronectin and neurons with a fibrin gel, and then place the fibrin gel with embedded fibronectin and neurons as a therapeutic device to bridge a severed nerve.

An additional embodiment is the fabrication of fabrics. For example, the biopolymer scaffold is built using silk, the strongest biological fiber known to man. The ability to control silk alignment at the nano/micro scale will result in fabrics with unique strength and other physical properties such as the ability to create engineered spider webs. Such engineered spider webs could be used for a multitude of applications such as, but not limited to, catching clots in the blood stream, removing (filtering) particulates from gases or fluids and ultra-light, ultra-strong fabrics for high-performance activities providing abrasion resistance, perspiration wicking and other properties.

### EXAMPLES

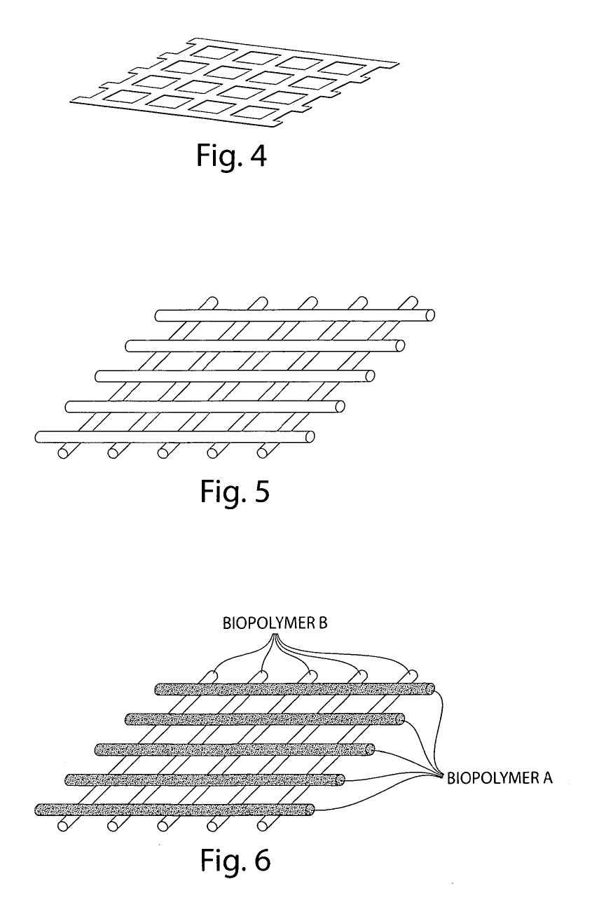

The structures in one possible fabrication process for creating the free-standing biopolymer scaffolds and structures are illustrated in FIGS. 1-4. As shown in FIG. 1, a transitional polymer [for example, Poly(N-Isopropylacrylamide (PIPAAm))] is coated onto a glass cover slip serving as a rigid substrate. As shown in FIG. 2, a biopolymer (e.g., the extracellular matrix protein, fibronectin) is microcontact printed onto the PIPAAm using PDMS stamps in a pattern dictated by the microstructures on the stamp. An example of the printed biopolymer pattern on the transitional polymer is illustrated in FIG. 3. This printing step may be repeated with different stamps and/or different biopolymers to create intricately patterned, multi-biopolymer layers. The PIPAAm, which has a lower critical solution temperature (LCST) of approximately 32° C., is dissolved by exposure to room-temperature de-ionized water. The free fibronectin scaffold or structure, illustrated in FIG. 4, is released as an integral structure with its shape preserved and now suspended in solution; the released structure can then be used for the desired application.

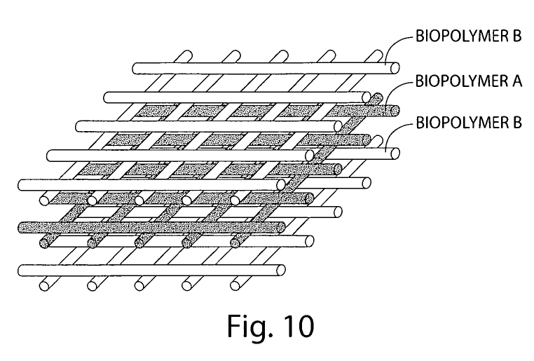

Representative examples of the types of biopolymer scaffolds that may be generated using the described methods are illustrated in FIGS. 5-10. As shown in FIG. 5, a scaffold can be generated where there is only a single biopolymer component. Although a grid-like net structure is illustrated in FIG. 5, nearly any interconnected network or isolated structures can be generated using microcontact printing stamps of appropriate design.

As shown in FIG. 6, a multiple-component scaffold including two or more biopolymers and/or a biopolymer at two or more concentrations/densities may be generated by multiple stampings prior to release of the scaffold from the transitional polymer. In this embodiment, two layers of parallel structures, wherein each layer is formed of a different biopolymer, are vertically stacked. Biopolymer A can be, e.g., laminin, while biopolymer B can be, e.g., fibronectin.

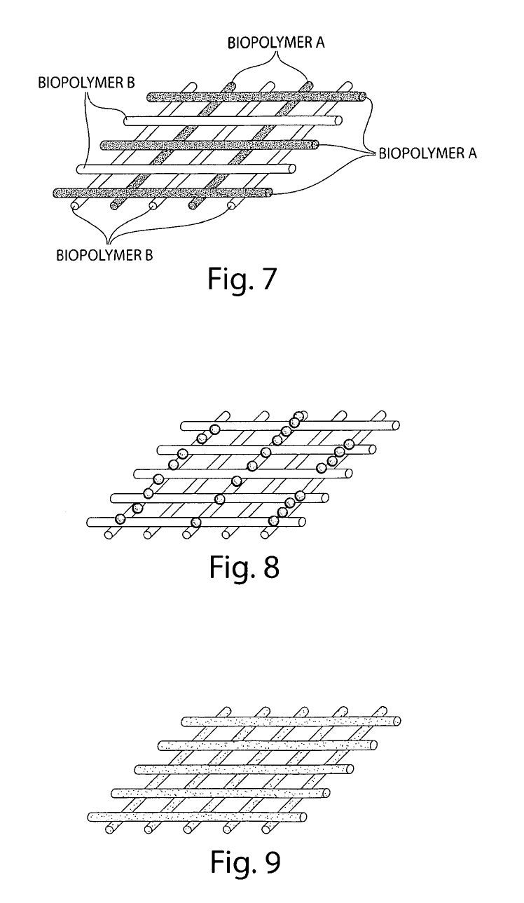

Spatially interdigitated multiple component biopolymer scaffolds, shown in FIG. 7, are generated via the process for producing the scaffold of FIG. 6, with the addition of careful spatial registration between stampings, such that intricate patterns are formed.

In another embodiment, growth factors and/or signaling molecules are incorporated into single- or multiple-component biopolymer scaffolds, as shown in FIG. 8. The growth factors and/or signaling molecules can be mixed in directly with the biopolymers, producing uniform density where patterned. Alternatively, the growth factors and/or signaling molecules can be patterned directly, creating unique concentration/density gradients to elicit specific cellular function, such as neuronal axon extension along a specific axis.

As shown in FIG. 9, multiple-component scaffolds can also be generated by direct mixing of two or more biopolymers into a mixed solution prior to stamping. In this embodiment, the composition of the scaffold is substantially uniform throughout.

Once released from the transitional layer, multiple biopolymer scaffolds of any type, such as any of those illustrated in FIGS. 5-9, can be stacked on top of one another to create three-dimensional scaffolds with nanometer-to-millimeter-to-centimeter spatial control and with control of the biopolymer composition down to the same nanometer scale (e.g., between 1 nm and 1 μm).

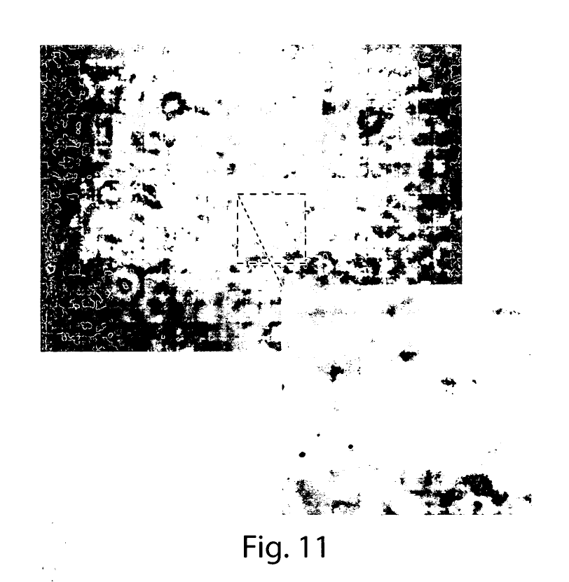

An image of patterned lines of fibronectin on PIPAAm after a two-step stamping process is provided in FIG. 11. The fibronectin is patterned as 20-μm-wide, 20-μm-spaced lines with the second stamping performed at a 90° rotation in orientation to the first stamping to create a grid-like pattern (see inset). The pattern is visible under 20× phase contrast, bright field illumination due to the slight difference in index of refraction of the PIPAAm and fibronectin pattern. This inspection serves as simple way to verify fidelity of the patterned biopolymer.

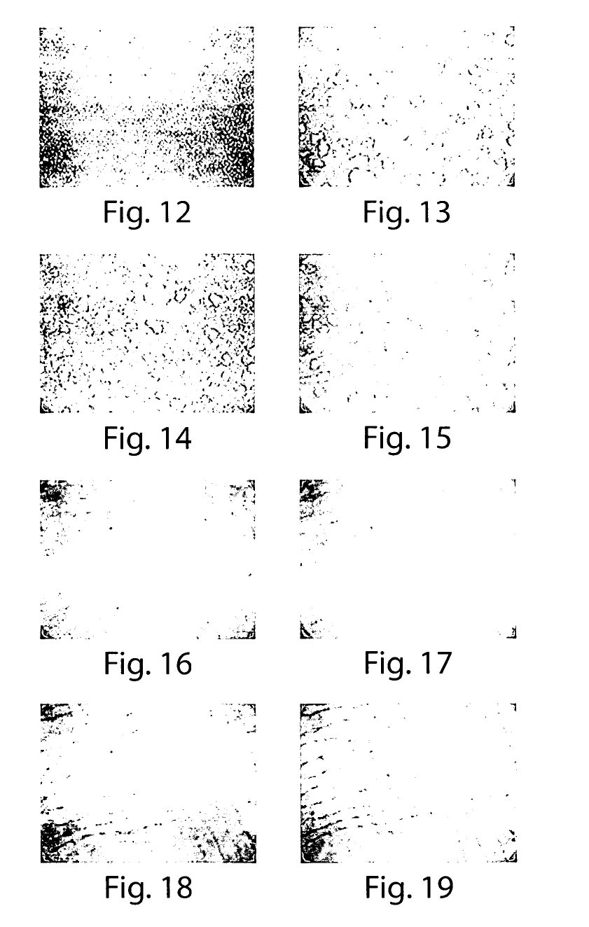

Sequential, time-lapse images are provided in FIGS. 12-19 for the release of a fibronectin ECM net based biopolymer scaffold from PIPAAm as the de-ionized water is cooled from 37° C. to 27° C. Initially at 37° C., the PIPAAm is in a solid, hydrophobic state, as shown in FIG. 12. As the PIPAAm cools below the LCST at −32° C., however, the PIPAAm becomes hydrophilic, concurrently swelling with water and dissolving into solution, as shown in FIGS. 13-15. As the PIPAAm dissolves, the fibronectin ECM net becomes visible and the flat lines of fibronectin collapse in forming interconnected tendrils, as shown in FIGS. 16-19.

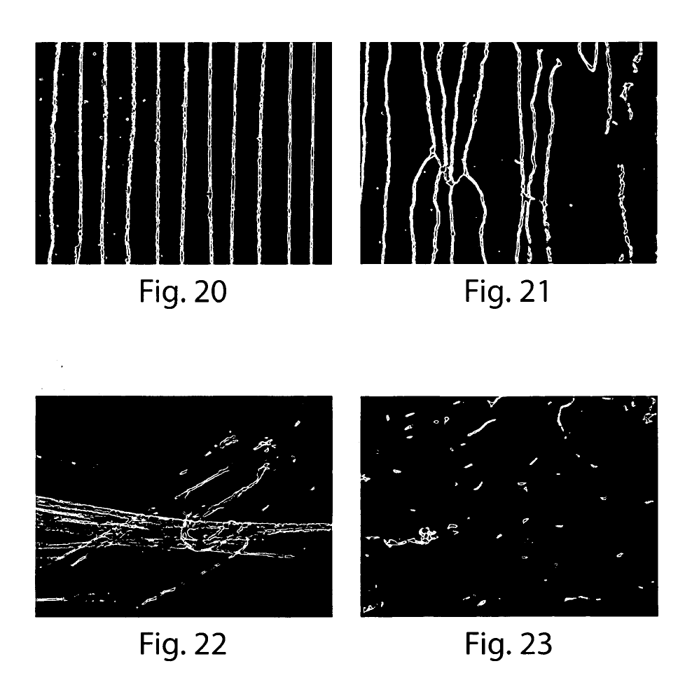

Examples of fibronectin structures after release from PIPAAm are illustrated in FIGS. 20-23. The fibronectin has been stained with fluorescently labeled antibodies to enhance visualization at 20× magnification. As shown in FIG. 20, lines of fibronectin originally 20 μm wide, 20 μm spaced have remained in near-parallel and substantially evenly spaced alignment relative to each other, but the originally flat lines have collapsed in and formed narrow tendrils (˜5 μm wide). As shown in FIG. 21, some lines are still interconnected by a second 90° stamping that was of poor fidelity creating cross connection in random positions. FIG. 22 shows a bundle of fibronectin line tendrils after release. Finally, the illustration of small fibronectin fragments shaped like triangles in FIG. 23 demonstrates that a multitude of independent structures with defined geometries can be produced.

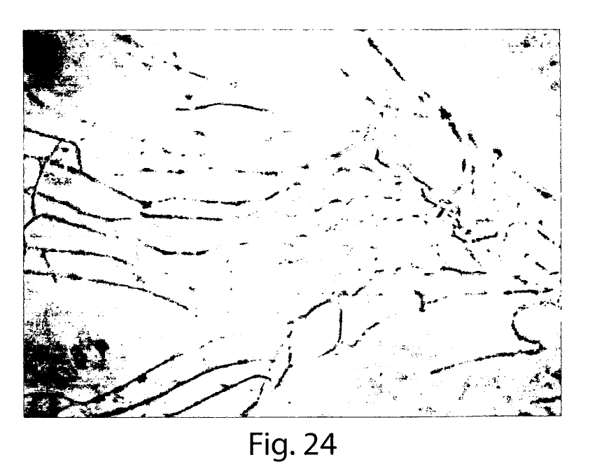

An example of a fibronectin ECM Net after release from PIPAAm, imaged at 20× magnification under phase contrast, bright field illumination, is illustrated in FIG. 24.

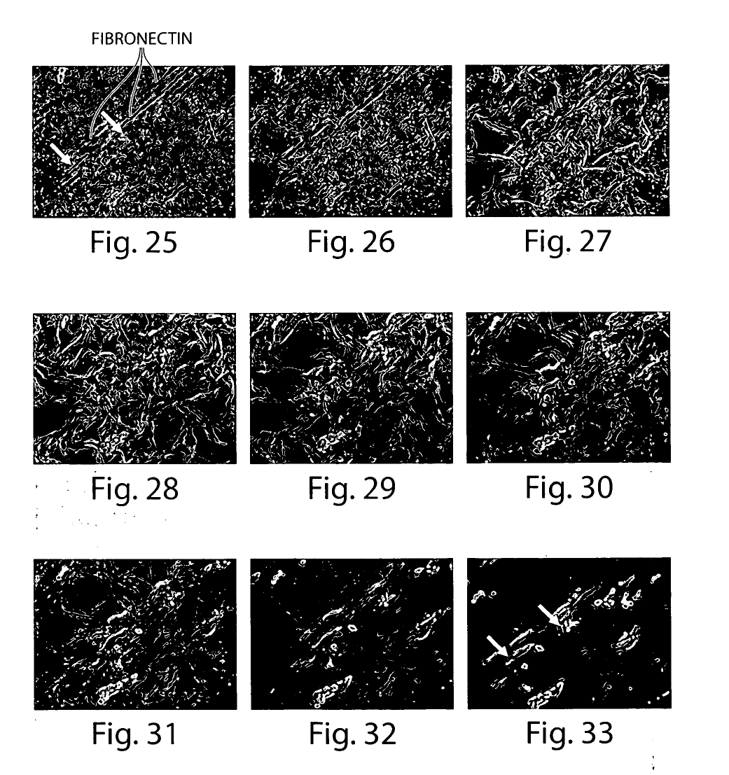

A sequential, time-lapse series showing the release of patterned lines of fibronectin and cardiomyocytes from PIPAAm is illustrated in FIGS. 25-33. This series demonstrates a method for patterning a biopolymer scaffold on a transitional polymer and then seeding cells on the scaffold prior to releasing the scaffold from the transitional polymer layer. In this example, fibronectin lines 20 μm wide and spaced at 10 μm were patterned on poly(N-Isopropylacrylamide) and then seeded with rat ventricular myocytes and cultured at 37° C. Once adhered, the sample was cooled to room temperature, which caused the PIPAAm to gradually expand and dissolve (in FIGS. 26-32), releasing the patterned myocytes into culture (in FIG. 33). This process verifies the ability to pattern myocytes directly on the patterned PIPAAm and the ability to generate myocyte structures that maintain their shape after release.

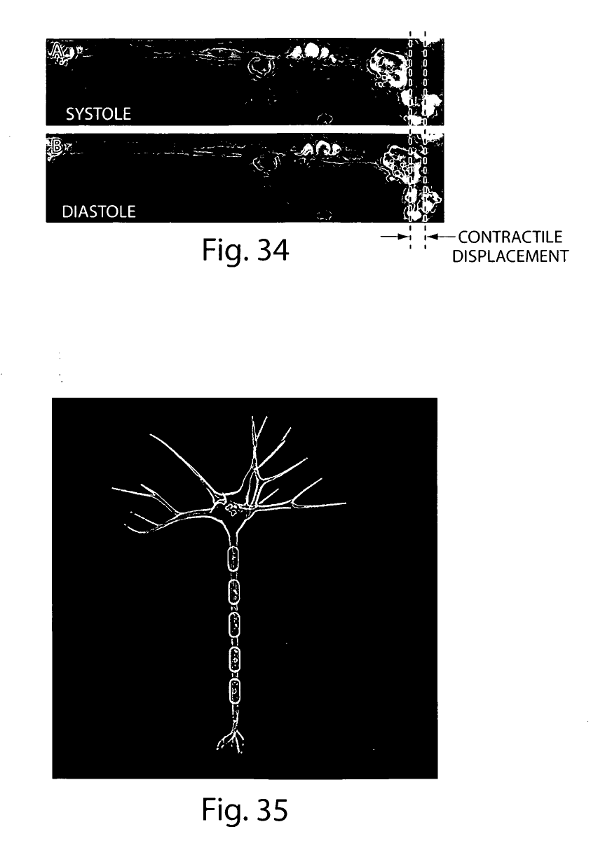

FIG. 34 demonstrates that the myocyte patterned into lines using a biopolymer scaffold, as described, above, and illustrated in FIGS. 25-33, is capable of generating functional myocyte constructs. Two frames are illustrated from a video of a few aligned, connected and contracting myocyte in (A) systole and (B) diastole. The displacement of the end of the myocyte strip is illustrated and represents a distance of approximately 10 μm.

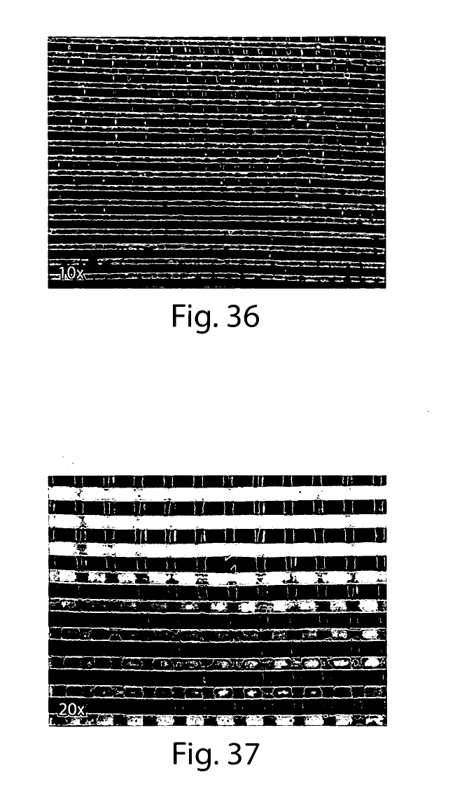

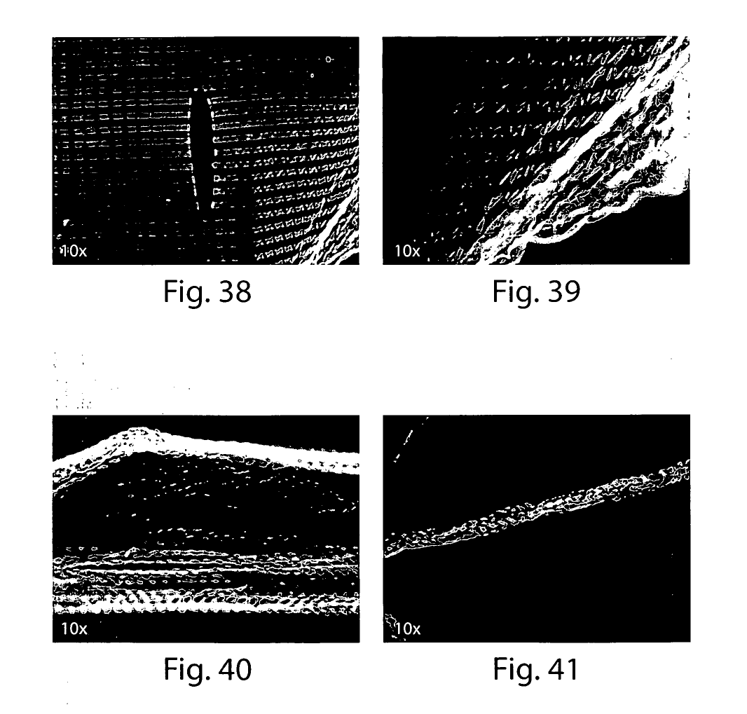

An example of a biopolymer scaffold created by micropatterning laminin (green, horizontal lines) and fibronectin (red, vertical lines) on PIPAAm spin coated on glass cover slips is illustrated in FIGS. 36-41. The images show the biopolymer scaffold after thermal release from the PIPAAm and demonstrate that a released scaffold can be composed of at least two different proteins; that the proteins adhere to each other (as shown in FIGS. 36 and 37); that the scaffold is defect tolerant to small tears (as shown in FIG. 38); that the scaffold rolls up in place, showing that is a free structure (as shown in FIGS. 39 and 40); and that the scaffold will curl into ribbons in places (as shown in FIG. 41). The vertical lines are 20 μm by 20 μm lines of laminin micropatterned on PIPAAm; and the horizontal lines are 20 μm by 20 μm lines of FN micropatterned on PIPAAm at 90° to the Laminin lines. ECM grids are stained at 37° C. with mouse α-FN and rabbit α-laminin concurrently for 1.5 hours, washed and then secondarily stained with goat α-mouse rhodamine and goat α-rabbit Alexa Fluor 488 concurrently for 1.5 hours.

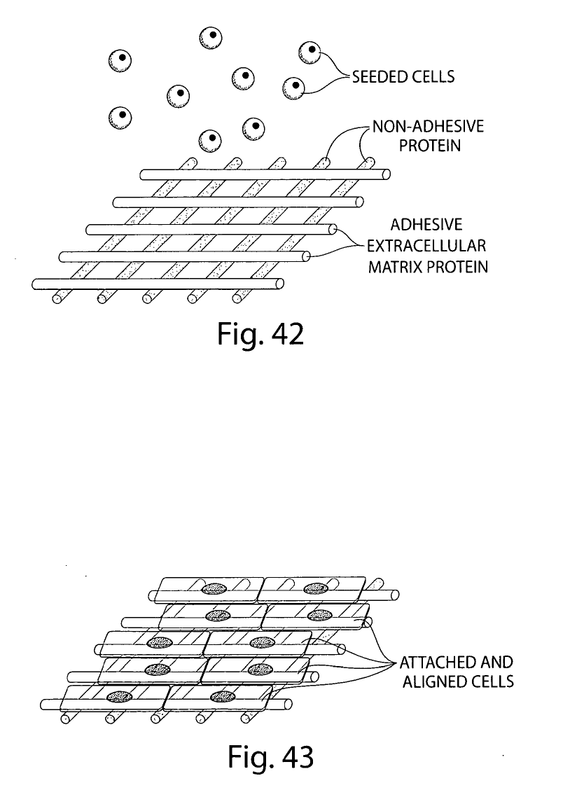

An example wherein a biopolymer scaffold design is used to create a monolayer thick anisotropic two-dimensional myocardium that directs the self assembly of cardiomyocytes along a single axis is illustrated in FIGS. 42 and 43. The free-standing polymer scaffold is supported by a frame (support system) during cell seeding and tissue formation. Seed cells (e.g., cardiomyocytes) are deposited onto a free-standing biopolymer scaffold, which includes vertical lines of a non-adhesive protein (such as bovine serum albumin) and horizontally oriented lines of an adhesive extracellular matrix protein (such as fibronectin) in FIG. 42. Meanwhile, FIG. 43 shows the oriented, adhesive extracellular matrix proteins directing the self-assembly of cells (e.g., cardiomyocytes to form two-dimensional myocardium) into an anisotropic tissue. The patterning of extracellular matrix proteins along a single (horizontal) axis supports cell elongation along this axis only. The orthogonal (vertical) lines of bovine serum albumin hold the biopolymer scaffold together while limiting lateral growth due to the non-adhesive qualities of bovine serum albumin to cells. Similar strategies in designing and building biopolymer scaffolds can be used, for example, to direct nerve growth in a similar manner for regeneration and repair, or to create oriented sheets of smooth muscle cells to repair vascular aneurysms.

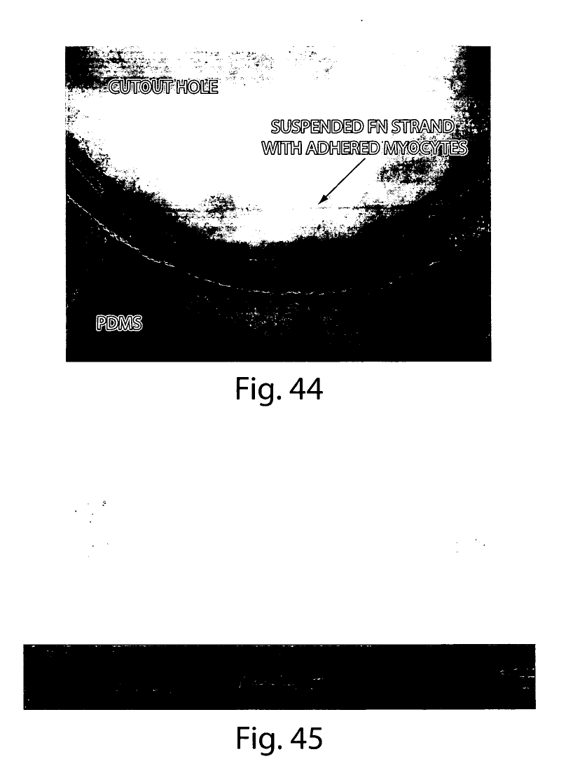

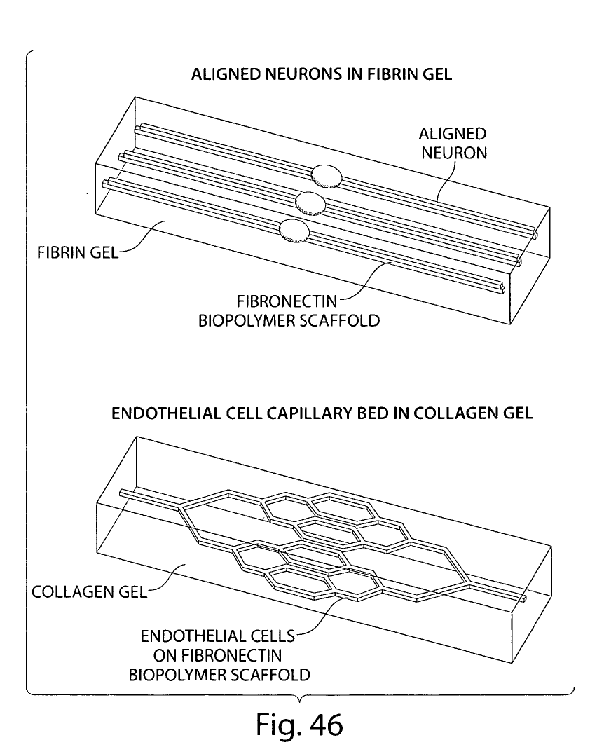

Finally, an example of a single strand of a biopolymer scaffold suspended across a hole cut into a PDMS film and with cardiomyocytes cultured thereon is illustrated in FIG. 44. A magnified view of the single strand is illustrated in FIG. 45. Cardiomyocytes were seeded onto the strand and cultured for 4 days. The cardiomyocytes on the biopolymer scaffold actively contracted, thereby demonstrating the ability to use the biopolymer scaffold for tissue engineering applications where cells are seeded onto the free-standing, biopolymer construct and form a functional tissue. This tissue engineered example resembles the chordea tendinea that control the open/closed state of heart valves.

In describing embodiments of the invention, specific terminology is used for the sake of clarity. For purposes of description, each specific term is intended to at least include all technical and functional equivalents that operate in a similar manner to accomplish a similar purpose. Additionally, in some instances where a particular embodiment of the invention includes a plurality of system elements or method steps, those elements or steps may be replaced with a single element or step; likewise, a single element or step may be replaced with a plurality of elements or steps that serve the same purpose. Moreover, while this invention has been shown and described with references to particular embodiments thereof, those skilled in the art will understand that various substitutions and alterations in form and details may be made therein without departing from the scope of the invention; further still, other aspects, functions and advantages are also within the scope of the invention. The contents of all references, including issued patents and published patent applications, cited throughout this application are hereby incorporated by reference in their entirety. The appropriate components and methods of those references may be selected for the invention and embodiments thereof.

What is claimed is:

1. A method for creating biopolymer structures:
providing a transitional polymer on a substrate;
depositing a biopolymer on the transitional polymer;
shaping the biopolymer into a structure having a selected pattern on the transitional polymer; and
releasing the biopolymer from the transitional polymer with the biopolymer's structure and integrity intact.

2. The method of claim 1, wherein the biopolymer is selected from extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, silks, and polyproteins.

3. The method of claim 1, wherein the biopolymer is selected from the group consisting of vitronectin, laminin, collagen, fibrinogen, silk, and silk fibroin.

4. The method of claim 1, wherein the biopolymer is deposited via soft lithography.

5. The method of claim 4, wherein the deposited structure includes features with dimensions of less than 1 micrometer.

6. The method of claim 4, wherein the biopolymer is printed on the transitional polymer with a polydimethylsiloxane stamp.

7. The method of claim 6, further comprising printing multiple biopolymer structures with successive, stacked printings.

8. The method of claim 7, wherein each biopolymer is a protein, and wherein different proteins are printed in different printings.

9. The method of claim 1, wherein the biopolymer is deposited via self assembly on the transitional polymer.

10. The method of claim 9, wherein the self assembly is selected from assembly of collage into fibrils, assembly of actin into filaments, and assembly of DNA into double strands.

11. The method of claim 1, wherein the biopolymer is deposited via vaporization of the biopolymer and deposition of the biopolymer through a mask onto the transitional polymer.

12. The method of claim 1, wherein the biopolymer is deposited via patterned photo-cross-linking on the transitional polymer.

13. The method of claim 12, wherein patterned light photo-cross-links the biopolymer in the selected pattern.

14. The method of claim 13, further comprising dissolving non-cross-linked biopolymer outside the selected pattern.

15. The method of claim 12, wherein patterned light changes the reactivity of the biopolymer via release of a photoliable group or via a secondary photosensitive compound in the selected pattern.

16. The method of claim 1, further comprising allowing the biopolymer to bind together via a force selected from hydrophilic, hydrophobic, ionic, covalent, Van der Waals, and hydrogen bonding or via physical entanglement.

17. The method of claim 1, where the biopolymer structure is released by applying a solvent to the transitional polymer to dissolve the transitional polymer or to change the surface energy of the transitional polyther, wherein the biopolymer structure is released into the solvent as a free-standing structure.

18. The method of claim 1, wherein the biopolymer is released by applying a positive charge bias to the transitional polymer, by allowing the transitional polymer to undergo hydrolysis, or by subjecting the transitional polymer to enzymatic action.

19. The method of claim 1, wherein the transitional polymer comprises poly(N-Isopropylacrylamide).

20. The method of claim 1, wherein the biopolymer is patterned as a mesh structure.

21. The method of claim 1, further comprising stacking a plurality biopolymer structures formed via the method of claim 1 to produce a multi-layer scaffold.

22. The method of claim 21, further comprising integrating living cells into the scaffold.

23. The method of claim 22, further comprising growing the living cells in the scaffold to produce three-dimensional, anisotropic myocardium.

24. The method of claim 22, further comprising growing the living cells in the scaffold to produce a replacement organ.

25. The method of claim 22, further comprising growing the living cells in the scaffold to produce consumable meat or produce with an engineered composition.

26. The method of claim 22, where the living cells are stem cells, further comprising growing the living cells in the scaffold where the structure, composition, ECM type, growth factors and/or other cell types assist in differentiation of stem cells into functional, engineered tissue to produce a replacement tissue or organ.

27. The method of claim 1, further comprising wrapping the biopolymer structure around a three-dimensional implant and then inserting the implant into an organism.

28. The method of claim 1, further comprising placing the biopolymer structure on or in a wound.

29. The method of claim 1, wherein the substrate has an elastic modulus is greater than 1 MPa.

30. The method of claim 1, wherein the substrate is selected from a glass cover slip, polystyrene, polymethylmethacrylate, polyethylene terephthalate film, gold and a silicon wafer.

31. A free-standing biopolymer structure comprising an integral pattern of the biopolymer having repeating features with a dimension of less than 1 mm and without a supporting substrate.

32. The free-standing biopolymer structure of claim 31, wherein the structure has repeating features with a dimension of 100 nm or less.

33. The free-standing biopolymer structure of claim 31, wherein the biopolymer structure comprises at least one biopolymer selected from extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, and combinations thereof.

34. The free-standing biopolymer structure of claim 31, further comprising cells seeded on the patterned biopolymer.

35. A free-standing biopolymer structure comprising an integral pattern of the biopolymer and molecular remnant traces of poly(N-Isopropylacrylamide).

36. A freestanding functional tissue structure comprising a flexible polymer scaffold imprinted with a predetermined pattern and cells attached to said polymer, said cells being spatially organized according to said pattern, wherein said cells are functionally active.

37. The structure of claim 36, wherein said cells are muscle cells selected from the group consisting of smooth muscle cells, striated muscle cells, and cardiac cells.

38. A composition comprising a plurality of freestanding tissue structures, each of said structures comprising a flexible polymer scaffold imprinted with a predetermined pattern, muscle cells and adipose cells attached to said polymer in spatially organized manner according to said pattern to yield an edible meat product, the texture, taste and/or nutritional content of said meat product being distinguished from a naturally-occurring meat.

39. A composition comprising a plurality of freestanding tissue structures, each of said structures comprising a flexible polymer scaffold imprinted with a predetermined pattern, plant cells attached to said polymer in spatially organized manner according to said pattern to yield an edible fruit or vegetable product, the texture, taste and/or nutritional content of said product being distinguished from a naturally-occurring fruit or vegetable.

40. A free-standing biopolymer structure comprising an integral pattern of the biopolymer having repeating features with a dimension of less than 1 mm and with a supporting frame during tissue formation.

41. The free-standing biopolymer structure of claim 40, wherein the structure has repeating features with a dimension of 100 nm or less.

42. The free-standing biopolymer structure of claim 40, wherein the biopolymer structure comprises at least one biopolymer selected from extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, silks, polyproteins (e.g., poly(lysine)) and combinations thereof.

43. The free-standing biopolymer structure of claim 40, further comprising cells seeded on the patterned biopolymer.

44. A free-standing biopolymer structure comprising an integral pattern of the biopolymer having repeating features with a dimension of less than 1 mm and embedded within a 3-dimensional gel.

45. The free-standing biopolymer structure of claim 44, wherein the structure has repeating features with a dimension of 100 nm or less.

46. The free-standing biopolymer structure of claim 44, wherein the biopolymer structure comprises at least one biopolymer selected from extracellular matrix proteins, growth factors, lipids, fatty acids, steroids, sugars and other biologically active carbohydrates, biologically derived homopolymers, nucleic acids, hormones, enzymes, pharmaceuticals, cell surface ligands and receptors, cytoskeletal filaments, motor proteins, and combinations thereof.

47. The free-standing biopolymer structure of claim 44, further comprising cells seeded on the patterned biopolymer before being embedded within a gel.

48. The free-standing biopolymer structure of claim 44, further comprising cells mixed in with the gel pre-curser thus being trapped within the gel when polymerized around the patterned biopolymer.

49. The free-standing biopolymer structure of claim 44, further comprising cells seeded after the patterned biopolymer is embedded within a gel.

50. The free-standing biopolymer structure of claim 44, wherein the biopolymer structure is embedded in a gel that comprises at least one biological hydrogel selected from fibrin, collagen, gelatin, elastin and other protein and/or carbohydrate derived gels or synthetic hydrogel selected from polyethylene glycol, polyvinyl alcohol, polyacrylamide, poly(N-isopropylacrylamide), poly(hydroxyethyl methacrylate) and other synthetic hydrogels, and combinations thereof.
